(19) United States
(12) Patent Application Publication
(10) Pub. No.: US 2018/0015020 A1
(43) Pub. Date: Jan. 18, 2018

(54) BLEACHING AGENTS HAVING REDUCED OIL-SEPARATION TENDENCY

(71) Applicant: Henkel AG & Co. KGaA, Duesseldorf (DE)

(72) Inventors: Udo Erkens, Willich (DE); Burkhard Mueller, Duesseldorf (DE)

(73) Assignee: Henkel AG & Co. KGaA, Duesseldorf (DE)

(21) Appl. No.: 15/547,067

(22) PCT Filed: Oct. 30, 2015
(86) PCT No.: PCT/EP2015/075201

§ 371 Date: Jul. 28, 2017

(30) Foreign Application Priority Data
Feb. 6, 2015 (DE) 10 2015 202 189.0

(51) Int. Cl. A61K 8/73; A61K 8/23; A61K 8/31; A61Q 5/08

(52) U.S. Cl. CPC A61K 8/731; A61Q 5/08; A61K 8/23; A61K 8/31; A61K 2800/48

(57) ABSTRACT

Agent for lightening keratin fibers and method for changing the color of keratinic fibers are provided herein. In an embodiment, an agent for lightening keratin fibers includes, relative to the weight thereof, a) from about 0.01 to about 5 wt % ethyl cellulose, b) from about 5 to about 70 wt % oil component(s), c) from about 1 to about 70 wt % peroxydisulphate(s) from the group of sodium peroxydisulphate and/or potassium peroxydisulphate and/or ammonium peroxydisulphate.

### CROSS-REFERENCE TO RELATED APPLICATION

This application is a U.S. National-Stage entry under 35 U.S.C. §371 based on International Application No. PCT/EP2015/075201, filed Oct. 30, 2015 which was published under PCT Article 21(2) and which claims priority to German Application No. 10 2015 202 189.0, filed Feb. 6, 2015, which are all hereby incorporated in their entirety by reference.

### TECHNICAL FIELD

The present disclosure relates to agents for oxidative color change in the field of cosmetics, which are particularly designed for lightening keratin fibers, particularly human hair.

### BACKGROUND

The oxidizing agents contained in bleaching compositions are able to lighten the hair fibers by the oxidative destruction of the melanin pigment in the hair itself. For a moderate bleaching effect it is generally sufficient to use hydrogen peroxide alone as the oxidizing agent—possibly with the addition of ammonia or other alkalizing agents, in order to achieve a more pronounced bleaching effect a mixture of hydrogen peroxide and peroxy-disulphate salts and/or peroxymonosulphate salts is used.

For stability reasons, commercial bleaching agents are usually offered in two separately packaged preparations which are mixed immediately before use to provide a finished application preparation. Commercially available bleaching agents usually include a liquid oxidizing agent preparation and a powder containing solid oxidizing agents. Alternatively, paste-like agents may be mixed with a liquid oxidizing agent preparation instead of the powder, in which case the problems of dust generation during manufacture and mixing are avoided. Products with other components are also available commercially.

Paste-like bleaching agents typically contain relatively large quantities of an inert oil, which can cause stability problems (settling of the solid oxidants out of the oil and separation of the oil component). Even if the peroxydisulphates have not yet fully settled, a concentration gradient may form in the package so that different portions of the packaging can produce inconsistent bleaching after mixing. To minimize these problems, a high degree of viscosity is desirable.

On the other hand, the viscosity of the bleaching paste must be low enough to enable it to be mixed well and quickly with the liquid oxidizing agent preparation. In the case of anhydrous bleaching agents, easy and homogeneous miscibility with the typically aqueous oxidizing agent preparation is a particularly important and technically complex requirement.

The resulting bleaching mixture must also be also fluid enough to enable it to be applied easily and uniformly, but thick enough not drip off the head or application tools used, such as brushes. In addition, the resulting bleaching mixture should not separate, since settling or phase separations are considered a quality defect by customers.

Often hair is not only to be lightened, but dyed at the same time. Bleaching pastes should therefore be readily miscible with mousse-like dyes. These colorants often contain direct dyes, so homogeneous mixing is essential for a uniform dyeing and lightening result.

WO 2009/134875 A1 describes bleaching agents that contain persulphate salts and an oil gel, which in turn includes oil(s) and certain polymers.

EP 1 034 777 A1 discloses agents for lightening keratin fibers which contain at least two separately packaged preparations (A) and (B), which are mixed immediately before use to create an application mixture, wherein the preparations (A) are oil-based and contain polymer(s) that form oleo- or lipogels. In this context, mixtures with natural polymers such as xanthan are also disclosed.

### BRIEF SUMMARY

Agent for lightening keratin fibers and method for changing the color of keratinic fibers are provided herein. In an embodiment, an agent for lightening keratin fibers includes, relative to the weight thereof, a) from about 0.01 to about 5 wt % ethyl cellulose, b) from about 5 to about 70 wt % oil component(s), c) from about 1 to about 70 wt % peroxydisulphate(s) from the group of sodium peroxydisulphate and/or potassium peroxydisulphate and/or ammonium peroxydisulphate.

In another embodiment, a method for changing the color of keratinic fibers is provided. In accordance with the method, at least two separately packaged preparations (A) and (B) are provided. Preparation (A) includes at least one persulphate and preparation (B) includes at least one oxidising agent. The preparations (A) and (B) are mixed to form an application mixture. The application mixture is applied to the fibers and is rinsed off again after a contact time. The preparation (A) includes a) from about 0.01 to about 5 wt % ethyl cellulose, b) from about 5 to about 70 wt % oil component(s), c) from about 1 to about 70 wt % peroxydisulphate(s) from the group of sodium peroxydisulphate and/or potassium peroxydisulphate and/or ammonium peroxydisulphate.

In another embodiment, an agent for lightening keratin fibers includes, relative to the weight thereof, a) from about 0.3 to about 1.5 wt % ethyl cellulose having formula (I)

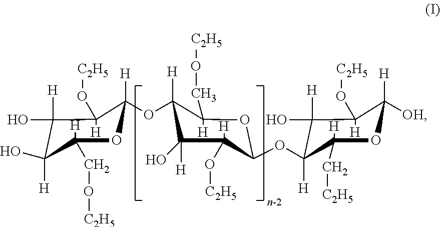

wherein n stands for integers from about 200 to about 250, b) from about 5 to about 70 wt % oil component(s), c) from about 12.5 to about 45 wt % peroxydisulphate(s) chosen from the group of sodium peroxydisulphate and/or potassium peroxydisulphate.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was to further improve the properties of bleaching agents, in particular resistance to oil separation should be increased. Miscibility with dye-containing formulations should also be improved.

It has been found that the use of certain copolymers achieves the abovementioned objects in both aqueous and anhydrous systems.

Desirable properties of the agent according to the present disclosure are stability in terms of settling and phase separation.

In a first embodiment, the object of the present disclosure is represented by agents for lightening keratin fibers, containing relative to the weight thereof
a) from about 0.01 to about 5 wt % ethyl cellulose,
b) from about 5 to about 70 wt % oil component(s),
c) from about 1 to about 70 wt % peroxydisulphate(s) from the group of sodium peroxydisulphate and/or potassium peroxydisulphate and/or ammonium peroxydisulphate.

Keratinous fibers or keratin fibers as well are understood to include animal fur, wool, feathers, and particularly human hair. Although the agents as contemplated herein are primarily designed to lighten keratin fibers, in principle there is nothing to prevent them from being used in other areas.

The first ingredient the agents as contemplated herein contain is ethyl cellulose.

Particularly preferred agents as contemplated herein are exemplified in that they contain from about 0.05 to about 7.5 wt %, preferably from about 0.1 to about 6 wt %, more preferably from about 0.15 to about 5 wt %, particularly preferably from about 0.2 to about 4 wt % and especially from about 0.3 to about 1.5 wt %, ethyl cellulose having formula

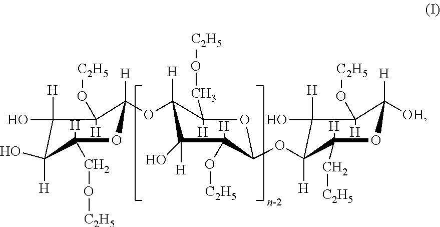

wherein n stands for integers from about 50 to about 500, preferably from about 100 to about 400, more preferably from about 150 to about 300, and particularly from about 200 to about 250.

For the ethyl cellulose(s) used in the agents as contemplated herein, the degree of substitution, that is to say the average number of etherised hydroxy groups per glucose unit is preferably between about 20 and about 60%. Particularly preferred agents as contemplated herein are exemplified in that they contain from about 0.05 to about 7.5 wt %, preferably from about 0.1 to about 6 wt %, more preferably from about 0.15 to about 5 wt %, particularly preferably from about 0.2 to about 4 wt % and especially from 0.3 to about 1.5 wt %, ethyl cellulose(s) whose degree of substitution is from about 45 to about 50%, preferably from about 46 to about 49.9%, more preferably from about 47 to about 49.8%, still more preferably from about 47.5 to about 49,7% and especially from about 48 t0 about 49.5%.

In combination with the ingredients b) and c), the ethyl cellulose(s) form agreeably viscous bleaching pastes which have excellent stability and drastically reduced oil separation. Moreover, their miscibility with dye-containing formulations and the with developer emulsion is significantly improved. Even the ready mixed bleaching agents manifest a viscosity which is very well suited to the application area and very stable.

As the second ingredient, the agents as contemplated herein contain from about 5 to about 70 wt % oil component(s). This/these oil(s) is/are preferably liquid at 25° C.

With regard to the cosmetic oils, a distinction is made between volatile and non-volatile oils. Non-volatile oils are is also understood to refer to those oils which have a vapour pressure less than 2.66 Pa (0.02 mmHg) at 20° C. and an ambient pressure of 1013 hPa. Volatile oils include those oils which have a vapour pressure of from about 2.66 Pa-about 40,000 Pa (from about 0.02 mm-about 300 mm Hg), preferably from about 10-about 12,000 Pa (from about 0.1-about 90 mmHg), particularly preferably from about 13 about 3,000 Pa, most particularly preferably from about 15-about 500 Pa at 20° C. and an ambient pressure of 1013 hPa.

Volatile cosmetic oils are usually selected from cyclic silicone oils with INCI name cyclomethicone. INCI name cyclomethicone is particularly understood to include cyclotrisiloxane (hexamethylcyclotrisiloxane) cyclotetrasiloxane (octamethylcyclotetrasiloxane), cyclopentasiloxane (decamethylcyclopentasiloxane) and cyclohexasiloxane (dodecamethylcyclohexasiloxane). These oils have a vapour pressure of from about 13-about 15 Pa at 20° C.

A cyclomethicone substitute that is preferred as contemplated herein is a mixture of C₁₃-C₁₆ isoparaffins, C₁₂-C₁₄ isoparaffins and C₁₃-C₁₅-alkanes having a viscosity in the range from about 2 to about 6 mPas at 25° C. and with a vapour pressure in the range from about 10 to about 150 Pa, preferably from about 100 to about 150 Pa at 20° C. Such a mixture is available commercially for example with the name SiClone SR-5 from Presperse Inc.

Other preferred volatile silicone oils are selected from volatile linear silicone oils, particularly volatile linear silicone oils having 2-10 siloxane units, such as hexamethyldisiloxane (L₂), octamethyltrisiloxane (L₃), decamethyltetrasiloxane (L₄), as are contained for example in the commercial products DC 2-1184, Dow Corning® 200 (0.65 cSt) and Dow Corning® 200 (1.5 cSt) from Dow Corning, and low molecular weight phenyl trimethicone having a vapour pressure of about 2000 Pa at 20° C., as is available commercially from GE Bayer Silicones/Momentive under the name Baysilone Fluid PD 5 for example.

Further preferred products as contemplated herein contain at least one volatile non-silicone oil. Preferred volatile non-silicone oils are selected from C₈-C₁₆-isoparaffins, especially from isononane, isododecane, isoundecane, isododecane, isotridecane, isotetradecane, isopentadecane and isohexadecane, and mixtures thereof. Preferred are C₁₀-C₁₃-isoparaffin mixtures, particularly those having a vapour pressure of from about 10-about 400 Pa, preferably from about 13-about 100 Pa at 20° C.

Particularly preferred for use as a cosmetic oil as contemplated herein are esters of linear or branched saturated or unsaturated fatty alcohols having 2-30 carbon atoms with linear or branched saturated or unsaturated fatty acids having 2-30 carbon atoms, which may be hydroxylated. Preferred are esters of linear or branched saturated fatty alcohols having 2-5 carbon atoms with linear or branched saturated or unsaturated fatty acids having 10-18 carbon atoms, which may be hydroxylated. Preferred examples thereof are isopropyl palmitate, isopropyl stearate, isopropyl myristate, 2-hexyldecyl stearate, 2-hexyldecyl laurate, isodecyl neopentanoate, isononyl isononanoate, 2-ethylhexyl palmitate, and 2-ethylhexyl stearate. Also preferred are isopropyl isostearate, isopropyl oleate, isooctyl stearate, isononyl stearate, isocetyl stearate, isononyl isononanoate, isotridecyl isononanoate, cetearyl isononanoate, 2-ethyl hexyl laurate, 2-ethylhexyl isostearate, 2-ethylhexyl cocoate, 2-octyldodecyl palmitate, butyloctanoic acid 2-butyloctanoate, diisotridecylacetate, n-butyl stearate, n-hexyl laurate, n-decyl oleate, oleyl oleate, oleyl erucate, erucyl oleate, erucyl erucate, ethylene glycol dioleate, ethylene glycol dipalmitate, n-hexyl laurate, n-decyl oleate, oleyl oleate, oleyl erucate, erucyl oleate, C₁₂-C₁₅-alkyl lactate and di-C₁₂-C₁₃-alkylmalate and the benzoic acid esters of linear or branched C₈₋₂₂ alkanols. Particularly preferred are benzoic acid-C₁₂-C₁₅ alkyl esters, available commercially for example as Finsolv® TN (C₁₂-C₁₅-alkyl benzoate) and benzoic acid isostearyl esters, available commercially for example as Finsolv® SB, 2-ethylhexyl benzoate, available commercially for example as Finsolv EB® and benzoic acid 2-octyldodecyl ester, available commercially for example as Finsolv® BOD.

The use of isopropyl esters of C₁₂-C₁₅ carboxylic acids, in particular the use of isopropyl myristate and particularly preferably mixtures of isopropyl myristate with C₁₀-C₁₃-isoparaffin mixtures, the latter preferably having a vapour pressure of from about 10-about 400 Pa at 20° C., has proved particularly advantageous.

A further particularly preferred ester oil is triethyl citrate. Further preferred products as contemplated herein contain triethyl citrate and at least one C₈-C₁₆-isoparaffin selected from isononane, isododecane, isoundecane, isododecane, isotridecane, isotetradecane, isopentadecane and isohexadecane and mixtures of these isoparaffins. Further preferred products as contemplated herein contain triethyl citrate and at least one C₈-C₁₆-isoparaffin selected from isononane, isododecane, isoundecane, isododecane, isotridecane and mixtures of these C₈-C₁₆-isoparaffins. Further preferred products as contemplated herein contain triethyl citrate and a mixture of isodecane, isoundecane, isododecane and isotridecane.

As used hereinafter, the term “triglyceride” means “glycerol triester”. Further non-volatile oils preferred as contemplated herein are selected from the triglycerides of linear or branched, saturated or unsaturated, optionally hydroxylated C₈-₃₀ fatty acids, provided they are liquid under normal conditions. Particularly suitable may be the use of natural oils such as soybean oil, cottonseed oil, sunflower oil, palm oil, palm kernel oil, linseed oil, almond oil, castor oil, corn oil, rapeseed oil, olive oil, sesame oil, safflower oil, wheatgerm oil, peach kernel oil and the liquid fractions of coconut oil and the like. Particularly preferred are synthetic triglyceride oils, in particular capric/caprylic triglycerides, e.g., the commercial products Myritol® 318 or Myritol® 331 (BASF/Cognis) with unbranched fatty acid radicals as well as glyceryl triisostearin and glyceryl(2-ethylhexanoate) having branched fatty acid radicals. Such triglyceride oils preferably make up less than about 50 wt % of the total weight of all cosmetic oils in the product as contemplated herein.

Further particularly preferred non-volatile non-silicone oils as contemplated herein are selected from the dicarboxylic acid esters of linear or branched C₂-C₁₀-alkanols, especially diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further particularly preferred non-volatile non-silicone oils as contemplated herein are selected from the symmetrical, asymmetrical or cyclic esters of carbonic acid with C₆-C₂₀-alcohols, such as di-n-caprylyl carbonate (Cetiol® CC) or di-(2-ethylhexyl) carbonate (Tegosoft DEC). On the other hand, esters of carbonic acid with C₁-C₅-alcohols, such as glycerol carbonate or propylene carbonate, are not suitable for use as a cosmetic oil.

Other oils that may be preferred as contemplated herein are selected from the esters of dimers of unsaturated C₁₂-C₂₂ fatty acids (dimer fatty acids) with monovalent linear, branched or cyclic C₂-C₁₈-alkanols or with polyvalent linear or branched C₂-C₆-alkanols. Particularly preferably, the total weight of dimer fatty acid esters is from about 0.5 -about 10 wt %, preferably from about 1-about 5 wt %, relative in each case to the weight of the entire water-in-oil emulsion, without taking into account the weight of the propellant.

Further cosmetic oils that are particularly preferred as contemplated herein are selected from non-volatile silicone oils. Preferred non-volatile silicone oils as contemplated herein are selected from linear polyalkyl siloxanes with a kinematic viscosity of at least about 5 cSt to about 2000 cSt at 25° C., particularly selected from linear polydimethylsiloxanes having a kinematic viscosity of from about 5 cSt to about 2000 cSt, preferably from about 10 to about 350 cSt, particularly preferably from about 50 to about 100 cSt at 25° C., such as those available commercially under the tradenames Dow Corning® 200 or Xiameter PMX from Dow Corning or Xiameter. Further preferred non-volatile silicone oils are phenyltrimethicones having a kinematic viscosity of from about 10 to about 100 cSt, preferably from about 15 to about 30 cSt at 25° C. and cetyldimethicones.

Preferred agents as contemplated herein contain at least one non-volatile silicone oil, which is preferably selected from linear polyalkylsiloxanes with a kinematic viscosity of from about 5 cSt-about 2000 cSt, preferably from about 10-about 350 cSt, particularly preferably from about 50-about 100 cSt at 25° C., particularly selected from linear polydimethylsiloxanes with a kinematic viscosity of from about 5 cSt-about 2000 cSt, preferably from about 10 to about 350 cSt, particularly preferably from about 50-about 100 cSt at 25° C., in a total amount of from about 0.1-about 30 wt %, preferably from about 1-about 24 wt %, particularly preferably from about 2-about 18 wt %, most especially preferably from about 4-about 10 wt %, relative to the weight of the total composition in each case.

Of these oils, some have proven particularly suitable, since they guarantee the physical and chemical stability of the bleaching agent pastes for long periods and are highly compatible with the other ingredients as contemplated herein. Preferred agents as contemplated herein are exemplified in that they contain from about 22.5 to about 70 wt %, preferably from about 25 to about 65 wt %, more preferably from about 27.5 to about 60 wt %, particularly preferably from about 30 to about 55 wt % and especially from about 32.5 to about 50 wt % oil(s) from the group of paraffin oil, polyisobutene, the alkyl benzoates, isopropyl palmitate, the C₁₄₋₂₂-alkanes, isononyl isononanoate.

The preparations as contemplated herein also comprise from about 1 to about 70 wt % peroxydisulphate(s) from the group of sodium peroxydisulphate and/or potassium peroxydisulphate and/or ammonium peroxydisulphate as a further essential ingredient.

In this context, agents which contain certain peroxydisulphates in narrower quantitative ranges have proven particularly suitable. Extremely preferred agents contain from about 2.5 to about 65 wt %, preferably from about 5 to about 60 wt %, more preferably from about 7.5 to about 55 wt %, particularly preferably from about 10 to about 50 wt % and most particularly from about 12.5 to about 45 wt % peroxydisulphate(s) from the group of sodium peroxydisulphate and/or potassium peroxydisulphate.

It is greatly preferable if the quantity of potassium peroxydisulphate is always kept at a significantly higher level than the quantity of any sodium peroxydisulphate and ammonium peroxydisulphate used. It has been found that as the potassium peroxydisulphate fraction of the total quantity of peroxydisulphates increases, the chemical and physical stability of the agents as contemplated herein increases. In preferred agents therefore, the weight ratio of potassium peroxydisulphate to sodium peroxydisulphate and ammonium peroxydisulphate is >about 2, preferably >about 5, more preferably >about 10, still more preferably >about 15 and particularly >about 20. This weight ratio is calculated by dividing the quantity of potassium peroxydisulphate in wt % by the sum of the quantities of sodium peroxydisulphate and ammonium peroxydisulphate in wt %.

Preferred agents as contemplated herein are exemplified in that the weight ratio of potassium peroxydisulphate contained in the agent to the sodium peroxydisulphate and ammonium peroxydisulphate contained in the agent >about 10:1, preferably >about 12.5:1, more preferably >about 15:1, particularly preferably >about 17.5:1, and especially >about 20:1.

Most especially preferred agents as contemplated herein contain from about 0 to <about 2.5 wt %, preferably from about 0 to <about 1 wt %, more preferably from about 0 to <about 0.5 wt %, particularly preferably from about 0 to <about 0.1 wt % and especially 0 wt % peroxydisulphates from the group of sodium peroxydisulphate and/or ammonium peroxydisulphate.

The agents as contemplated herein may contain at least one natural polymer as a further ingredient. Cellulose derivatives which are used as thickeners for example may serve as the natural polymer. Examples are agar, carrageenan, alginates, xanthan gum, karaya gum, ghatti gum, tragacanth, scleroglucan gum or gum arabic, alginates, pectins, polyoses, guar gum, carob bean flour, linseed gums, dextrans, pectins, starch fractions and derivatives such as amylose, amylopectin and dextrins, gelatin and casein, and cellulose derivatives such as methylcellulose, carboxyalkylcelluloses such as carboxymethylcellulose, and hydroxyalkylcelluloses such as hydroxyethyl cellulose.

Natural polymers from these substance classes are commercially available and are offered for example under the trade names Deuteron®-XG (anionic heteropolysaccharide based on (3-D-glucose, D-mannose, D-glucuronic acid, Schoener GmbH), Deuteron®-XN (non-ionogenic polysaccharide, Schoener GmbH), Protanal RF 6650 alginate (sodium alginate, FMC Biopolymer), Cekol (cellulose gum, Kelco), Kelzan (xanthan biopolymer, Kelco) xanthan FN (xanthan biopolymer, Jungbunzlauer), Keltrol e.g. Keltrol CG-T (xanthan biopolymer, Kelco) or Keltrol CG-SFT (xanthan biopolymer, Kelco).

In a preferred embodiment as contemplated herein, the agents contain xanthan gum. Preferred as contemplated herein are those xanthans which yield transparent preparations upon swelling. Particularly preferred is the use of the xanthan biopolymer which is marketed by Kelco under the trade name Keltrol CG-SFT.

In a preferred embodiment, an agent as contemplated herein contains from about 0.1 to about 5 wt %, preferably from about 0.5 to about 4 wt %, more preferably from about 1 to about 3 wt %, particularly preferably from about 1.25 to about 2.5 wt % and in particular from about 1.5 to about 2 wt % xanthan.

The agents as contemplated herein may preferably contain fatty alcohols as volumizers, wherein the fatty alcohols are preferably selected from the group of arachyl alcohol (eicosane-1-ol), gadoleyl alcohol ((9Z)-eicos-9-en-1-ol), arachidonic alcohol ((5Z, 8Z,11Z,14Z)-eicosa-5,8,11,14-tetraene-1-ol), heneicosyl alcohol (heneicosane-1-ol), behenyl alcohol (docosane-1-ol), erucyl alcohol ((13Z)-docos-13-en-1-ol) and brassidyl alcohol ((13E)-docosene-1 -ol).

These long-chain fatty alcohols have a chain length of at least 20 C-atoms. Within this group, particularly long-chain fatty alcohols have proven most particularly suitable. In a particularly preferred embodiment, an agent for bleaching and/or lightening keratin fibers is exemplified in that it contains arachyl alcohol (eicosane-1-ol).

In a particularly preferred embodiment, an agent for bleaching and/or lightening keratin fibers is exemplified in that it contains arachyl alcohol (eicosane-1-ol) and/or behenyl alcohol (docosane-1-ol).

It has further been found that it is advantageous if the long-chain fatty alcohols, especially arachyl alcohol (eicosane-1-ol) and/or behenyl alcohol (docosane-1-ol), are contained in the agents of the present disclosure in certain quantity ranges. Preferred agents as contemplated herein contain one or more long-chain fatty alcohols (a) from the group arachyl alcohol (eicosan-1-ol), gadoleyl alcohol ((9Z)-eicos-9-ene-1-ol), arachidonic alcohol ((5Z,8Z,11Z,14Z)-eicosa-5,8,11,14-tetraene-1-ol), heneicosyl alcohol (heneicosane-1-ol), behenyl alcohol (docosane-1-ol), erucyl alcohol ((13Z)-docos-13 -ene-1-ol) and brassidyl alcohol ((13E)-docosene-1-ol) in a total quantity from about 0.3 to about 3.4 wt %, preferably from about 0.4 to about 2.6 wt %, more preferably from about 0.5 to about 1.8 wt %, and particularly preferably from about 0.6 to about 0.9 wt %—relative to the total weight of the application-ready product.

In a most particularly preferred embodiment, an agent as contemplated herein is exemplified in that it contains as fatty alcohol(s) arachyl (eicosane-1-ol) and/or behenyl alcohol (docosane-1-ol) in a total quantity from about 0.3 to about 3.4 wt %, preferably from about 0.4 to about 2.6 wt %, more preferably from about 0.5 to about 1.8 wt %, and particularly preferably from about 0.6 to about 0.9 wt %—relative to the total weight of the application-ready product.

Besides the specific long chain fatty alcohols having a chain length of at least 20 carbon atoms, the agent as contemplated herein may also contain other, shorter-chain fatty alcohols having a chain length of 12 to 18 carbon atoms. Suitable shorter-chain fatty alcohols with a saturated C12-C18-alkyl chain are for example dodecane-1-ol (dodecyl alcohol, lauryl alcohol), tetradecane-1-ol (tetradecyl alcohol, myristyl alcohol), hexadecane-1-ol (hexadecyl alcohol, cetyl alcohol, palmityl alcohol) and octadecane-1-ol (octadecyl alcohol, stearyl alcohol). A suitable shorter-chain fatty alcohol with an unsaturated C12-C18-alkyl chain is for example (9Z)-octadec-9-ene-1-ol (oleyl alcohol).

Cetylstearyl alcohol is particularly suitable for physical and chemical stabilization.

Very particularly preferred agents as contemplated herein contain from about 1 to about 15 wt %, preferably from about 2 to about 12.5 wt %, more preferably from about 3.5 to about 11 wt %, particularly preferably from about 4 to about 10 wt % and especially from about 5 to about 7.5 wt % cetearyl alcohol.

Furthermore, the bleaching agent may contain alkalizing agents. Preferred alkalizing agents are for example ammonia, alkanolamines, basic amino acids and inorganic alkalizing agents such as alkaline (earth) metal hydroxides, alkaline (earth) metal metasilicates, alkaline (earth) metal phosphates and alkaline (earth) metal hydrogen phosphates. Suitable metal ions are preferably lithium, sodium and/or potassium.

Inorganic alkalizing agents that are usable as contemplated herein are preferably selected from sodium hydroxide, potassium hydroxide, calcium hydroxide, barium hydroxide, sodium phosphate, potassium phosphate, sodium silicate, potassium silicate, magnesium silicate, sodium carbonate and potassium carbonate. Particularly preferred are silicates.

Alkalizing agents that are usable as contemplated herein are preferably selected from alkanolamines from primary, secondary or tertiary amines with a C₂-C₆-alkyl base body which supports at least one hydroxyl group. Particularly preferred alkanolamines are selected from the group of 2-aminoethane-1-ol (monoethanolamine), 3-amino-propane-1-ol, 4-aminobutane-1-ol, 5-aminopentane-1-ol, 1-aminopropane-2-ol (monoisopropanolamine), 1-aminobutane-2-ol, 1-aminopentane-2-ol, 1-aminopentane-3-ol, 1-aminopentane-4-ol, 2-amino-2-methyl-propanol, 2-amino-2-methylbutanol, 3-amino-2-methyl propane-1-ol, 1-amino-2-methyl propane-2-ol, 3-amino-propane-1,2-diol, 2-amino-2-methylpropane-1, 3-diol, 2-amino-2-ethyl-1,3-propanediol, N,N-dimethyl ethanolamine, methylglucamine, triethanolamine, diethanolamine and triisopropanolamine. Particularly preferred alkanolamines are monoethanolamine, 2-amino-2-methyl-propanol and triethanolamine.

The basic amino acids that may be used as alkalizing agents as contemplated herein are preferably selected from the group of L-arginine, D-arginine, D/L-arginine, L-lysine, D-lysine, D/L-lysine, L-ornithine, D-ornithine, D/L-ornithine, L-histidine, D-histidine and/or D/L-histidine. L-arginine, D-arginine and/or D/L-arginine are particularly preferably used as the alkalizing agent as contemplated herein.

Some customers find the intense odor of ammonia troubling or irritating. Although ammonia is a preferred alkalizing agent, preferred application-ready preparations as contemplated herein may therefore be those which contain no ammonia. Preferred alkalizing agents for preparations which contain no ammonia are monoethanolamine, 2-amino-2-methyl-propanol and triethanolamine.

Agents that are particularly preferred as contemplated herein are exemplified in that they contain from about 0 to <about 0.1 wt %, preferably from about 0 to <about 0.05 wt %, more preferably from about 0 to <about 0.01 wt %, particularly preferably from about 0 to <about 0.001 wt % and especially 0 wt % ammonia.

If the application-ready mixtures contain alkalizing agents, as contemplated herein preparations are preferred which contain alkalizing agent in a quantity of from about 0.05 to about 20 wt %, particularly from about 0.5 to about 10 wt %, relative to the total weight of the application-ready agent in each case.

The compositions as contemplated herein may also contain at least one further bleach booster which is different from the inorganic persalts.

Compounds which release aliphatic peroxycarboxylic acids containing preferably 1 to 10 C-atoms, particularly 2 to 4 C-atoms and/or optionally substituted perbenzoic acid under conditions of perhydrolysis may be used as bleach boosters. Substances bearing O- and/or N-acyl groups having the indicated number of carbon atoms and/or optionally substituted benzoyl groups are suitable. Preferred are multiply acylated alkylenediamines, particularly tetraacetyl-ethylene diamine (TAED), acylated triazine derivatives, in particular 1,5-diacetyl-2,4-dioxyhexahydro-1,3,5-triazine (DADHT), acylated glycolurils, particularly tetraacetyl glycoluril (TAGU), N-acylimides, in particular N-nonanoylsuccinimide (NOSI), acylated phenol sulphonates, particularly n-nonanoyl- or isononanoyloxybenzene sulphonate (n- or iso-NOBS), carboxylic acid anhydrides, particularly phthalic anhydride, acylated polybasic alcohols, especially triacetin, ethylene glycol acetate and 2,5-diacetoxy 2,5-dihydrofuran.

The compositions of the present disclosure are stable in terms of viscosity and settling shortly after their preparation. The quantities of the ingredients are preferably adjusted to each other in such manner that the completed compositions having a viscosity suitable for the respective product category after a short rest period following production. Particularly preferred agents are exemplified in that 24 h after preparation they have a viscosity (Brookfield RV-2, Helipath, Spindle TF, 4 rpm, 60 s) of from about 100 Pas to about 10,000 Pas (from about 10⁵ mPas to about 10⁷ mPas), preferably from about 150 Pas to about 7,500 Pas (from about 1.5×10⁵ mPas to about 7.5×10⁶ mPas), more preferably from about 200 Pas to about 5,000 Pas (from about 2×10⁵ mPas to about 5×10⁶ mPas) and in particular from about 250 Pas to about 2,000 Pas (from about 2.5×10⁵ mPas to about 2×10⁶ mPas) at 25° C.

A second object as contemplated herein is a method for changing the color of keratinic fibers, in which at least two separately packaged preparations (A) and (B), of which preparation (A) contains at least one persulphate and preparation (B) contains at least one oxidizing agent, are mixed to form an application mixture, this is applied to the fibers and is rinsed off again after a contact time, wherein preparation (A) contains
a) from about 0.01 to about 5 wt % ethyl cellulose,
b) from about 5 to about 70 wt % oil component(s),
c) from about 1 to about 70 wt % peroxydisulphate(s) from the group of sodium peroxydisulphate and/or potassium peroxydisulphate and/or ammonium peroxydisulphate.

The application-ready agents are prepared immediately prior to application to the hair by mixing the two compositions (A) and (B) and optionally a third composition (C) and/or further preparations. When application-ready agents including more than two preparations mixed to form a finished application mixture, it may be unimportant whether two preparations are mixed together first and then added and the third preparation is only added afterwards, or whether all preparations are combined and then mixed at the same time. The mixing can be carried out by stirring in a bowl or a beaker or by shaking in a sealed container.

In this context, the term “immediately” is understood to be a period from a few seconds to an hour, preferably up to 30 min, particularly up to 15 minutes.

The agents as contemplated herein are used in a method for lightening keratin fibers, particularly human hair, in which the agent is applied to the keratin fibers, left on the fibers at a temperature from room temperature to 45° C. for a contact time of from about 10 to about 60 minutes, and then rinsed with water again or washed out with a shampoo.

The contact time of the application-ready lightening agent is preferably from about 10 to about 60 min, particularly from about 15 to about 50 min, particularly preferably from about 20 to about 45 min. During the time the agent is in contact with the fibers it may be advantageous to support the bleaching process by supplying heat. The heat may be supplied by an external heat source such as a hot air blower, or particularly in the case of well, especially for a hair lightening on living subjects, using the subjects' own body temperature.

In the last alternative, the portion to be lightened is typically covered with a hood. An exposure phase at room temperature is also contemplated herein. The temperature during the contact time is preferably between about 20° C. and about 40° C., especially between about 25° C. and about 38° C. The lightening agents yield good lightening results even at physiologically tolerable temperatures of below about 45° C.

After the exposure time has ended, the remaining lightening preparation is rinsed out of the hair with water or a cleaning agent. Cleaning agents that may be used include in particular commercial shampoo, in which case in particular the cleaning agent may be dispensed with the cleaning agent and the rinsing process can be carried out with tap water if the lightening agent has a strongly tenside-containing carrier.

The preferred embodiments of the first object as contemplated herein also apply mutatis mutandis to the second object as contemplated herein.

Regarding the viscosity of the application mixture of the agents as contemplated herein and the developer emulsion and further components, methods are preferred in which the application mixture has a viscosity (Brookfield RV-2, Helipath, spindle TF, 4 rpm, 60 s) of from about 1 Pas to about 100 Pas (from about 10³ mPas to about 10⁵ mPas), preferably from about 5 Pas to about 80 Pas (from about 5×10³ mPas to about 8×10⁴ mPas), more preferably from about 10 Pas to about 65 Pas (from about 10⁴ to about 6.5×10⁴ mPas) and particularly from about 10 Pas to about 50 Pas (from about 10⁴ mPas to about 5×10⁴ mPas) at 25° C. 24 hours after preparation.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An agent for lightening keratin fibers, comprising relative to the weight thereof
a) from about 0.01 to about 5 wt % ethyl cellulose,
b) from about 5 to about 70 wt % oil component(s),
c) from about 1 to about 70 wt % peroxydisulphate(s) from the group of sodium peroxydisulphate and/or potassium peroxydisulphate and/or ammonium peroxydisulphate.

2. The agent according to claim 1, comprising from about 0.05 to about 7.5 wt % ethyl cellulose having formula (I) 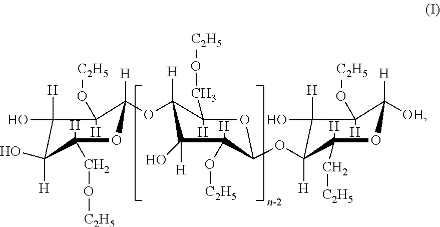
wherein n stands for integers from about 50 to about 500.

3. The agent according to either claim 1, comprising from about 0.05 to about 7.5 wt % ethyl cellulose(s) whose degree of ethoxylation is from about 45 to about 50%.

4. The agent according to claim 1, comprising from about 22.5 to about 70 wt % oil(s) chosen from the group of paraffin oil, polyisobutene, the alkyl benzoates, isopropyl palmitate, isohexadecane, isododecane, isononyl isononanoate, or combinations thereof.

5. The agent according to claim 1, comprising from about 1 to about 15 wt % cetearyl alcohol.

6. The agent according to claim 1, comprising from about 2.5 to about 65 wt % peroxydisulphate(s) from the group of sodium peroxydisulphate and/or potassium peroxydisulphate.

7. The agent according to claim 1, comprising from about 0 to about <0.1 wt % ammonia.

8. The agent according to claim 1, wherein after preparation, the agent has a viscosity (Brookfield RV-2, Helipath, Spindle TF, 4 rpm, 60 s) of from about 100 Pas to about 10,000 Pas (from about 10⁵ mPas to about 10⁷ mPas).

9. A method for changing the color of keratinic fibers, in which at least two separately packaged preparations (A) and (B), of which preparation (A) comprises at least one persulphate and preparation (B) comprises at least one oxidizing agent, wherein the preparations (A) and (B) are mixed to form an application mixture, wherein the application mixture is applied to the fibers and is rinsed off again after a contact time, and wherein preparation (A) comprises
a) from about 0.01 to about 5 wt % ethyl cellulose,
b) from about 5 to about 70 wt % oil component(s),
c) from about 1 to about 70 wt % peroxydisulphate(s) from the group of sodium peroxydisulphate and/or potassium peroxydisulphate and/or ammonium peroxydisulphate.

10. The method according to claim 9, wherein the application mixture has a viscosity (Brookfield RV-2, Helipath, spindle TF, 4 rpm, 60 s) of from about 1 Pas to about 100 Pas (from about 10³ mPas to about 10⁵ mPas).

11. The agent according to claim 1, comprising from about 0.3 to about 1.5 wt %, ethyl cellulose having formula (I) 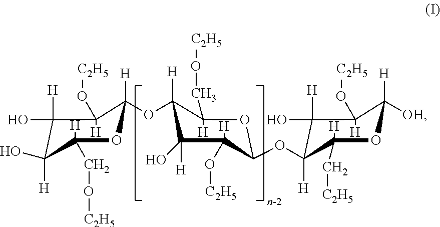
wherein n stands for integers from about 50 to about 500.

12. The agent according to claim 1, comprising from about 0.05 to about 7.5 wt %, ethyl cellulose having formula (I) 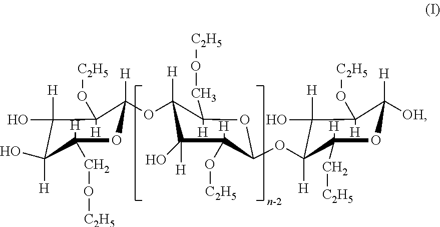
wherein n stands for integers from about 200 to about 250.

13. The agent according to claim 1, comprising from about 0.3 to about 1.5 wt %, ethyl cellulose(s) whose degree of ethoxylation is from about 45 to about 50%.

14. The agent according to claim 1, comprising from about 0.05 to about 7.5 wt % ethyl cellulose(s) whose degree of ethoxylation is from about 48 to about 49.5%.

15. The agent according to claim 1, comprising from about 32.5 to about 50 wt % oil(s) chosen from the group of paraffin oil, polyisobutene, the alkyl benzoates, isopropyl palmitate, isohexadecane, isododecane, isononyl isononanoate, or combinations thereof.

16. The agent according to claim 1, comprising from about 5 to about 7.5 wt % cetearyl alcohol.

17. The agent according to claim 1, comprising from about 12.5 to about 45 wt % peroxydisulphate(s) chosen from the group of sodium peroxydisulphate and/or potassium peroxydisulphate.

18. The agent according to claim 1, comprising from 0 wt % ammonia.

19. The agent according to claim 1, wherein after preparation, the agent has a viscosity (Brookfield RV-2, Helipath, Spindle TF, 4 rpm, 60 s) of from about 250 Pas to about 2,000 Pas (from about 2.5×10⁵ mPas to about 2×10⁶ mPas) at 25° C.

20. An agent for lightening keratin fibers, comprising relative to the weight thereof
a) from about 0.3 to about 1.5 wt % ethyl cellulose having formula (I) 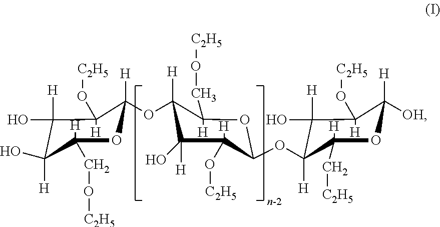
wherein n stands for integers from about 200 to about 250,
b) from about 5 to about 70 wt % oil component(s),
c) from about 12.5 to about 45 wt % peroxydisulphate(s) chosen from the group of sodium peroxydisulphate and/or potassium peroxydisulphate.
